(19) United States
(12) Patent Application Publication
(10) Pub. No.: US 2006/0184250 A1
(43) Pub. Date: Aug. 17, 2006

(54) Cement-less type artificial joint stem with the use of composite material

(76) Inventors: Shunichi Bandoh, Gifu (JP); Masaru Zako, Osaka (JP)

Correspondence Address: Apex Juris, 13194 Edgewater Lane Northeast, Seattle, WA, 98125, US

(21) Appl. No.: 10/545,082

(22) PCT Filed: Oct. 9, 2003
(86) PCT No.: PCT/JP03/13009

§ 371 Date: Aug. 8, 2005

(51) Int. Cl. A61F 2/36; A61F 2/28

(52) U.S. Cl. 623/23.32; 623/23.5; 623/23.35

(57) ABSTRACT

This invention provides cement-less type artificial joint stems with the use of complex material which can be connected to bone without using cement, does not get loose over a long period of time, has excellent durability, and has appropriate external form and stiffness to meet the condition of each patient. Stem 1 with the use of composite material inserted in the insertion hole 8 which is penetrated into the bone 7 and fixed to the bone 7 without using cement, has the external form of the epiphysis which fits the internal form of the insertion hole 8, has the main part 3 with changing stiffness so that in the neighborhood of the boundary between the epiphysis and diaphysis, stiffness becomes lower as approaching toward the diaphysis, and possesses a neck part 2 to place a spherical head in an artificial joint provided at the proximal end of the main part.

### BACKGROUND OF THE INVENTION

### Field of the Invention

This invention relates to cement-less type artificial joints and in particular relates to artificial joint stems that comprise composite material.

It has long been known that an artificial joint made to imitate a joint is inserted when a damaged joint is removed due to a broken bone. As one example of this artificial joint, FIG. 10 shows a structure of a conventional total hip prosthesis used for a hip prosthesis. This total hip prosthesis 100 consists a socket 102 fixed to a pelvis 101, a spherical head 104 equivalent to a femoral head of a femur (103) and a stem 105 embedded in the femur 103.

As shown in the figure, the socket 102 and the head 104 make a pair and have a function of a spherical bearing. This socket 102 consists of synthetic resins such as high-density polyethylene, and the spherical head 104 comprises ceramics like zirconia or cobalt alloy. Such socket 102 and the head 104 have been improved in durability with many modifications in recent years so that they can maintain the functions longer than life expectancy of many patients who undergo total hip arthroplasty, and the focus has been shifted from the socket 102 and the head 104 to the stem 105 to prolong the life of the total hip prosthesis 100.

The stem is often made of metal, and titanium alloy such as cobalt alloy and Ti6A1—4V is mainly used, considering the strength and effect on the human body.

As a method of fixing the stem to the femur, adhesive called cement-type has been used so far, and a cement-type total hip prosthesis stem using the method will be described below based on FIGS. 11-15. FIG. 11 is a top view of examples of the conventional cement-type total hip prosthesis stem made of metal. FIG. 12 (A) shows the condition before the cement-type total hip prosthesis stem is placed, and FIG. 12(B) is a sectional view of the condition after the stem is placed in the femur. FIG. 13 is a sectional view of the internal structure of the proximal side epiphysis part of the femur. FIG. 14 is an enlarged sectional view of the internal structure of bone. Also, FIG. 15 (A) is a graph, showing the relationship between the modulus ratio of bone and the average porosity of bone, and FIG. 15(B) is a graph, showing the relationship between the thicknesswise compression ratio of bone and the average porosity of bone.

FIG. 11 shows various types of cement-type total hip prosthesis 105a-105d. These external forms are generally simple with straight lines, circles and circular arcs, and there are no problems although the external forms of the stems 105a-105d are simple because the adhesive is filled in the medullary canal constituting complex internal forms.

The method of fixing the cement-type total hip prosthesis stem to the femur 103 will be described below based on FIG. 12. First, spongy cancellous bone and bone marrow are removed from the medullary canal of the femur 103 with the use of a tool called broach, and an insertion hole 107 to insert the stem 105e is formed. Next, a boneplug 108 is embedded at the bottom of the insertion hole 107, and adhesive or cement 109 with two kinds of resin, base resin and hardener which are mixed at the predetermined ratio respectively is filled in the insertion hole 107 (see A). Then, the stem 105e is inserted in the insertion hole 107 and fixed to the femur 103 as the cement 109 hardens (see B).

In the epiphysis of the femur 103 where the stem is fixed, as shown in FIG. 13, the interior is fully filled with a spongy cancellous bone 110, and the cancellous bone 110 gradually decreases as approaching from the epiphysis 112 to the lower side of the diaphysis 113, and the interior of the diaphysis 113 is abbreviation cavities. Such bone structure is made by the force affecting as distributed loads on the spherical femoral head at the tip of the epiphysis 112 and is fairly rational in terms of dynamics.

Further, describing the bone structure based on FIG. 14, the outermost layer of bone has a compact bone 111, and the compact bone 111 is a part with high bone density and high strength. Meanwhile, the interior of the compact bone 111 is the spongy cancellous bone 110 with more refined cavities as approaching toward the center of bone, and the cancellous bone 110 has a weaker structure than that of the compact bone 111.

Therefore, regarding the strength characteristic of bone, as shown in FIG. 15(A) and FIG. 15(B), as the average porosity of bone (cavity ratio per unit area) increases, its modulus of elasticity and compressive strength both decrease. For that reason, bone has a structure with decreasing modulus of elasticity and compressive strength as approaching toward the center away from the outer layer. As to the cement-type total hip prosthesis stem, the stem 105 is fixed to the femur 103 by impregnating the cement 109 within the refined cavities of the cancellous bone 110.

In this way, regarding the cement-type total hip prosthesis stem, the stem 105 is fixed to the femur 103 by hardening the cement 109, so the stem 105 can be fixed to the femur 103 for a fairly short time, which has an advantage in rehabilitating early for patients who undergo replacement operation with the total hip prosthesis 100. Therefore, it is particularly effective for elderly patients who are confined to bed for a long time and concerned with negative effects on other functions including motor function.

However, the cement-type uses two kinds of resin, base resin and hardener as the cement 109, and if they are not mixed enough, or the mixture ratio is inaccurate, unreacted monomer resin components which are not polymerized would remain and have harmful effects on the human body through the melt-out, and it is a source of causing various damages to the human body. Therefore, there is hesitation in using the cement-type to the youth with a long life expectancy.

Also, as to the cement-type, the stem 105 is fixed to the cancellous bone 110 of the femur 103 through the cement 109, and since the stiffness and strength of the cancellous bone 110 are not enough, the adhesive property to the stem 105 gets worse due to the weight of the stem 105, and the stem 105 gets loose or moves downward, called a sinking-down phenomenon. Especially when the sinking-down phenomenon occurs, the spherical stem 105 creates circumferential hoop stress like severing bone. Then, when the bone is cracked, patients suffer from the pain over a long period of time since there is no way to treat it so far.

As for the total hip prosthesis, the cement-type requires re-operation at a rate of five to twenty percent within ten years, but it is difficult to pull the stem 105 with the cement-type out of bone, and the re-operation itself is not easy.

Now, the cement-less type, fixing the stem 105 to the femur 103 without the use of cement, has been developed, and the following explains the conventional cement-less total hip prosthesis stem with the use of the cement-less type, based on FIG. 16-FIG. 18. FIG. 16 is a top view of the embodiment of the conventional cement-less type total hip prosthesis stem. FIG. 17(A) is an enlarged view of the principal part of the convex portion on the side of the stem, and FIG. 17(B) is a fragmentary sectional view of the further enlarged sectional view. FIG. 18 is a sectional view of the conventional cement-less type total hip prosthesis stem fixed to the femur and cut in the axial direction, which is a different embodiment from that of FIG. 16.

As shown in FIG. 16, the conventional cement-less total hip prosthesis stem is made of metal such as titanium alloy which is the same as cement-type, and there are various forms in stems 105f-105j as shown in the figure, and as to the external forms of these stems 105f-105j, the part below neck 115 to fix the head 104 is somewhat bigger compared to the cement-type stems 105a-105e, but the forms as a whole are simple with the use of curves between straight lines. Compared to the cement type stems 105a-105e, the cement-less type stems 105f-105j have forms such that the gap between the external surface and internal surface of the insertion hole 107 of the stem 105 penetrated into the femur 103 narrows.

The cement-less type stem 105 is fixed to the femur 103, using growth of bone within the femur 103, and the gap between the internal surface of the insertion hole 107 and the external surface of the stem 105 narrows as the stem 105 is driven into the insertion hole 107 and bone grows from the internal surface of the insertion hole 107 toward the external surface of the stem 105, and thereby fixing the stem 105 to the femur 103.

As to this cement-less type stem 105, there is no adverse affect on the human body through the melt-out of the unreacted monomer in the cement 109 since the cement 109 is not used. Therefore, the cement-less type stem 105 can be also used to young patients. Moreover, in a re-operation because the stem 105 can be pulled out of bone with relative ease, it helps save trouble in re-operation.

However, the cement-less type fixes the stem 105 as bone grows, narrowing the gap between the bone and the stem 105, and it takes several months until the bone fills the gap, and the stem 105 is firmly fixed, and then patients need a rehabilitation period, which prolonged a period of patients' hospitalization, imposing a burden on patients. Moreover, due to a long period of hospitalization it was difficult to adopt the method to elderly people who were concerned with negative effects on other functions such as motor function.

Given this situation, in order for patients to rehabilitate, the convex portion 116 (concavity and convexity portion) is set up on the surface of the stem 105 so that the stem 105 can be fixed to the extent that patients do not have trouble living in the early stage of the postoperative period, and the stem 105 is mechanically connected to bone with the anchoring effect of the convex portion 116.

FIG. 17(A) and FIG. 17(B) are enlarged views of the convex portion 116 for the conventional cement-less type total hip prosthesis stem, and as shown in the figures, the stem 105 can be fixed to some extent in the early stage of patients' postoperative period as being mechanically connected to bone with concavity and convexity on the surface of the stem 105 and set-in structure of minute wedges or screws between the stem 105 and the bone. The size in the concavity and convexity of the convex portion 116 is very small, and various shapes are suggested.

Moreover, in addition to the mechanical joint, a chemical joint method is also suggested as the convex portion 116, and for instance, crystal of hydroxyapatite, the main component of bone, is attached to the surface of the stem 105 with adhesive or the like, and the stem 105 is fixed to the femur 103 by chemically binding hydroxyapatite of the stem 105 and by growing bone. The one with either a mechanical joint or chemical joint, or both has been suggested.

In this way, by setting up the convex portion 116 on the cement-less stem 105, the initial fixation can be achieved to some extent in the early stage of postoperative period, which could relieve some of the burden from patients who were hospitalized for a long time.

However, in the case of the stem 105 also, it was hard to say the initial fixation was perfect. Also, in the case of these cement-less type stems 105f-105j, the joint between the stem 105 and bone is only partially connected to the compact bone 111 with high bone strength and mostly connected to the cancellous bone 110 with low bone strength, and thereby the joint strength between the stem 105 and bone being weak, and the stem 105 got loose by repetitive loads from the stem 105.

Also, the conventional stem 105 is made of metal such as cobalt alloy and titanium alloy, and because these alloys are difficult to cut, it is very hard to process the convex portion 116 with microscopic convexo-concave on the surface of the stem 105, which made the stem 105 very expensive.

Moreover, these alloys are excellent in corrosion resistance, and because it is difficult to apply adhesive surface treatment to the surface to form electrically neutral and stable oxide coating for adhesion of hydroxyapatite's crystal, the bonding strength of the hydroxyapatite is not stable and the hydroxyapatite exfoliates, which, as a result, creates a problem that the stem 105 gets loose.

Also, because the external form of the stem 105 is simple, it does not fit the internal form of the medullary canal, the load to the femur 103 is concentrated, and thereby becoming a source of pain and breakdown of bone through forcibly driving the stem 105 into the medullary canal. Regarding the elderly with weak bone strength and patients with osteoporosis, because they cannot bear such operation in which the stem 105 is driven into the femur 103 with a hammer, the cement-less stems 105f-105j could not be adopted.

In order to solve these drawbacks, a new cement-less type stem has been suggested. FIG. 18 shows the cement-less type stem, and the stem 105k is called custom made, and it is to provide the stem 105k having an external form which fits the internal form of the medullary canal 117 in the femur 103 of patients whom the stem 105k is implanted.

The custom-made stem 105 k is taken pictures of each section in the two-dot chain line shown in FIG. 18 with a ultrasonic tomography photo device or the like, and numerical data is made, combining these images in three dimensions with three dimension CAD, and the external form of the stem 105 k is processed based on the numerical data with a numerically-controlled processing machine (NC, CNC), and then the surface is finished by hand.

As shown in FIG. 18, because the external form of the stem 105k fits the internal form of bone, and the gap between the stem and bone is small, the stem 105k is fixed to bone in the early stage of the postoperative period, which can relieve patients' burden. Also, since the stem can be connected with the compact bone 111 with high bone strength, fixation of the stem 105 is strengthened, preventing the stem 105 from getting loose.

However, as to the custom-made stem 105 k, as shown in the section perpendicular to the axis in FIG. 19, it proves that the part touching the internal surface of the medullary canal 117 is small in the circumferential direction. Especially the part of the epiphysis 112 of the proximal side of the femur 103 touching the internal surface of the medullary canal 117 is significantly small. On the other hand, the distal side, the contacting part is getting larger as approaching toward the diaphysis 113. Here, the proximal side of the femur 103 means the side of the hip joint, and the distal side means the side of the knee joint.

Although it tries to make the external form of the stem 105k fit the internal form of the medullary canal 117 as much as possible, the workability of the machine work for the external form of the stem 105k and the subsequent finish processing is required. To be more precise, generally when a three dimensional form is machined, the cutting tool for cutting the form uses a hemispheric-tipped ball-end mill, and with the ball-end mill, it cannot get a flat face only by the machine work, which leaves a trail like a furrow called sculpheight.

Therefore, it is necessary to smooth the surface by undercutting the sculpheight by hand after the machine work, but the metal used for the stem 105 such as titanium alloy is difficult to cut, and the finishing requires very hard work. Therefore, the cement-less type stem made of titanium alloy became very expensive. Moreover, when convexo-concave is formed on the stem 105 to fit the internal form of the medullary canal 117, the finishing work would become more difficult, and it was too costly to adopt the kind.

When designing the external form of the stem 105, one tries not to form convexo-concave on the surface, ensuring that the stem 105 does not get caught when inserting the stem in the medullary canal 117. Therefore, as shown in FIG. 19, because the internal form of the medullary canal 117 is complex in the proximal side of the femur 103, the external form of the stem 105 k cannot correspond to the internal form, reducing the part that contacts with the stem 105 k (see section Z1-section Z8-section in the figure). Meanwhile, because the internal form of the medullary canal is simple in the distal side, it can easily correspond to the external form of the stem 105k, expanding the area that contacts with the stem 105 k (see section Z9-section Z13-section in the figure).

There is a term, Fit and Fill, to describe the relationship between the stem and the medullary canal. Fit means the contact ratio to the cortical bone, which is the ratio of the length of the cortical bone touching the stem to the entire circumference of the medullary canal in a section perpendicular to the axis of bone. Fill means the filling ratio in the medullary canal of the stem, which is the ratio of the section area of the stem to the area of the medullary canal in a section perpendicular to the axis of bone.

The higher Fit and Fill is, the better the accessibility of the stem and bone and the stronger force is transmitted from the stem to the bone. Therefore, as shown in FIG. 19, because in the conventional stem 105k, Fit and Fill is low in the proximal side of the femur 103 and Fit and Fill is high in the distal side, with larger contacting area with bone, or the distal side where Fit and Fill is high receives more force coming from the stem 105 k to the femur 103.

As shown in FIG. 13, the ossein that constitutes the compact bone 111 and the cancellous bone 110, that is the trabecular bone, is formed to continuously extend to a particular direction, its strength increased in this particular direction and thus in the structure of orthotropic anisotropy. This structure is similar to that of bamboo and wooden board of straight grain. This trabecular bone extends out from bone's external form to the internal side in the epiphysis part 112, but in the diaphysis 113 the trabecular bone is formed along with the external form. This means that the epiphysis 112 is strong against the perpendicular force toward the bone's surface, and the diaphysis 113, conversely, is relatively weak against the perpendicular force toward bone's surface.

From the above, for the diaphysis 113, that is the distal side, there was a risk of a bone being destroyed when a large amount of force is transferred from the stem 105 since bone in this region is weak against the sidling force. Therefore, it is desirable to stabilize stem in the epiphysis (proximal side). That is, the best relationship between the stem and the medullary canal is expected in such ways that the fit and fill is high in the epiphysis section (proximal side) and the fit and fill is low in the diaphysis (distal side).

As such, it is known in the traditional system 105 that porous coating of titanium alloy is applied on the proximal side surface of the stem 105 in order to increase the conjugation of bone in the proximal side, and that fixing is not to be done in the distal side by reducing the conjugation with bone through mirror finishing the tip part of stem 105 locating in the distal side. Hereafter, the fixing in the proximal side and the fixing in the distal side are called the proximal fixing and the distal fixing respectively.

However, as shown in FIG. 19, the fit and fill is low in the proximal side and the contacting area is small, and thus there are areas where force from the stem 105 is applied to bone and other areas where the force is not applied, which results in the stress shielding. This stress shielding, deriving from bone's physiological behavior, is a phenomenon in which bone thickens in the section where force applies and, conversely, bone becomes thin in the section where force does not apply. In this way, bone becomes thin in the section where a force from the stem 105k does not apply, reducing the conjugation with the stem 105k and causing the stem 105k to become loose.

Also, as shown in FIG. 19, the stem 105k turns easily in the stem 105k because the contacting area between bone and the non-circular cross section in the proximate side—that is, the section matching the internal form of the medullary canal 117—is minimal, and because the cross section is a near circular form in the distal side. As a result, rotation and fixation of the system 105k had not been satisfactory.

Moreover, stainless alloy such as high corrosive resistant cobalt alloy and titanium alloy is used in the above-mentioned stem 105k. If the high corrosive resistant oxide film is removed through abrasion of the surface of the stem 105 by micro motion in the contacting area with bone resulting from the stainless alloy being embedded in the body for a long period of time, the micro opening called corrosion pit is generated from the body fluid because the salinity in the body is the same as that of seawater. There has been a case reported, in which the metal fatigue is caused from the corrosion pit, fracturing the stem.

As such, various materials are suggested as the stem's raw material to replace metals. Some composite materials are among the suggestions. FIG. 20 indicates the nature of the strength (fatigue strength) of the composite materials. First, while the fatigue strength of the titanium alloy 118a decreases gradually as the loading applies repeatedly, the composite material 119, especially in the case of the carbon fiber reinforced plastic (CFRP), has an excellent durability, in which its fatigue strength rarely decreases even if the loading applies repeatedly. The symbol 118b, shown by the dotted line in the figure, indicates the titanium alloy when it is macerated in the seawater.

For example, it has been suggested to make the center of the stem metallic and its outer side wrapped around by the composite materials such as FRP (fiber reinforced plastic). In U.S. Pat. No. 4,892,552, Japanese unexamined patent publication bulletin 5-92019, and published Japanese translations of PCT international publication for patent applications 6-500945, it is suggested to manifacture the stem using the carbon fiber reinforced plastic. The stems in these proposals attain the same stiffness as metal by using the carbon fiber reinforced plastic, and unlike metal, harmful substances do not melt out in the body by making the plastic that is macerating into fiber harmless to human body.

However, none of the above inventions have been in practical use in the current status. That is to say, it has failed to make the center of the stem metallic and its external side wrapped around with FRP since the stem becomes loose in the early postoperative period, resulting from micro motion between the FRP and bone or between the FRP and the center of the metallic section. The cause of this failure is thought to be the stem's bending stiffness only applies to the center of the metallic section, making the overall bending stiffness low, and the distribution of stress in the contacting area with bone is concentrated in the both ends, leading to the occurrence of the micro motion since the stem cannot resist to the stress.

Also, U.S. Pat. No. 4,892,552 claims that from the sheet-shaped laminate made from carbon fiber impregnated with resin, coupons are cut out in a way that the carbon fiber's direction is parallel to the external form and other coupons are cut out in a way that the carbon fiber's direction is 45°, and these two types of coupons are piled up alternately and heat and pressure are applied to it to form a bloc, and the stem is manifactured by machining in which the bloc is scraped off. It is merely substituting metal with the composite material. While avoiding the harmful substance to melt out, it does not solve any other problems.

Furthermore, the unexamined patent publication bulletin 5-92019 claims the system having the first-direction strength support with reinforcing fiber in the longitudinal direction of the stem outside of the intermediate part that is hollow and the second-direction strength support with reinforcing fiber in the 45° from the longitudinal direction of the stem further outside. In this stem, the first-direction strength support deals with bending stiffness and the second-direction strength support deals with torsional stiffness with a structure utilizing the characteristics of composite material. However, the second-direction strength support located outside the stem is manifactured by wrapping the strip-shaped reinforcing fiber. With this method it is difficult to attain the external shape that fits the internal shape of the medullary canal, necessitating the coating layer further outside of the second-direction strength support, and the stem may get loose since the stress is concentrated in the both ends of the coating layer.

Moreover, the published Japanese translations of the PCT international publication for the patent applications 6-500945 claims the system having the core in the center with fiber located in the same direction as the longitudinal direction of the stem, and the filling material that is not fiber-reinforced outside the core, and the sheath with fiber arranged spirally outside the filling material. This system also cannot prevent the stem from getting loose, similar to the above-mentioned unexamined patent publication bulletin 5-92019.

The conventional systems cited above had common problems. It was the problem of concentration of stress caused by connecting the stem and bone. FIG. 21 explains the concentration of stress in a patterned form. FIG. 21(A) indicates the condition of stress on the adhesive joint when the members of the same stiffness are glued together. In this situation, the average stress applied on the adhesive joint between the member 120 and the member 121 is smaller than the simplified average stress calculated by simply dividing the compressive loading by the adhesive area, and the stress is applied mainly on both ends of the adhesive joints (indicated with dash line in the figure). On the other hand, the compressive stress of the member 120 and the member 121 gradually decreases by shear stress applied to the adhesive joint as getting toward the left in the figure and becomes zero at the left-end section (indicated with dashed lines in the figure).

Also FIG. 21(B) indicates the condition of stress on the adhesive joint when members of different stiffness are adhered. In this example, the member 121 in FIG. 21(A) is replaced by the member 122 with high stiffness. The stress is particularly concentrated at the right-end section of the adhesive joint, and the degree of stress is greater than that of FIG. 21(A) (indicated with dashed lines in the figure). Also, compressive stress is drastically reduced from the right-end section of the adhesive joint. We know from the above that the loading is transferred intensively at the one end of the adhesive joint when one member's stiffness is high.

Furthermore, FIG. 21(C) indicates the condition of stress on the adhesive joint when the length of adhesive joint in the example FIG. 21(B) is shortened. In this case, the average stress applied to the adhesive joint increases to the extent the adhesive area becomes smaller, yet the amount of stress concentration decreases and the total stress concentration does not change (indicated with dashed lines in the figure). Also, while compressive stress drastically decreases from the right-end section of the adhesive joint, high stress is maintained through the left-end section to the extent the adhesive section shortens (indicated with a dashed lines in the figure).

As shown in FIG. 21(A) and FIG. 21(B), we know hat the stress is concentrated at the end points of the adhesive section. That is, the stress concentration occurs at the both ends of connecting the point between the stem and bone. In particular, when comparing the stiffness of the stem and bone, the metallic stem made from titanium alloy is equivalent to the example in FIG. 21(B) and (C) since its stiffness is greater than that of bone, and a high loading concentration applies at the ends of the connecting section, starting the separation of the stem and bone from this section which leads to the stem to become loose.

Given the above factors, the method in FIG. 21(D) can be considered as a method to alleviate the occurrence of stress concentration at the ends of the adhesive joint. For the member 123, the taper section 124 is provided on the side opposite of the adhesive joint of the member 123, varying the thickness in the half way through the connecting section. As such, the stiffness of the member 123 decreases on the way to the right-end section, and extended to the right-end section while keeping the stiffness low. In this case, stress concentration drops drastically, becoming close to the average stress of the adhesive joint (indicated with dashed lines in the figure). Also, the distribution of compressive stress is similar to FIG. 21(C) (indicated with a dashed lines in the figure). Making the member 123 in such a form may reduce overall adhesive stress while keeping the member's overall compressive stress.

As a result, in the example of FIG. 21(D), the stress concentration is reduced while concentrating the stress at the adhesive section other than the ending points, and thus the separation of the adhesive section can be controlled even if the stress is concentrated.

That is, making the relationship between the stem and bone like FIG. 21(D) enables the stress concentration at the diaphysis to be transferred to epiphysis, and to control the occurrence of stress shielding since a high compressive stress is maintained at the adhesive section in its entirety. Also, the adhesive section is equivalent to the cancellous bone, and the separation of the cancellous bone from the stress concentration can be controlled at the end points of the connecting section with the stem.

However, the conventional system is manifactured from materials that are difficult to cut such as titanium alloy, and it was impossible to process in the hollow section, and thus the method in FIG. 21(D) cannot be applied to the conventional metallic stem.

In the example in FIG. 21(D), the member's thickness is varied as a means to change the stiffness. But for the composite material, the stiffness can also be changed by changing the direction of the composite material's fiber, in addition to the thickness of the member. Also, it is good to change both the thickness and the fiber's direction.

As such, considering the above situation, the invention can provide a cement-less type artificial joint stem with the use of composite material connecting to bone without using cement, not becoming loose over a long period of time, excellent in durability, and having appropriate external form and stiffness to each patient.

### SUMMARY OF THE INVENTION

In order to solve the above issue, the cement-less type artificial joint stem with the use of composite material in the invention is structured such that “the cement-less type artificial joint stem is inserted in an insertion hole which is penetrated into bone and fixed to the bone without using cement, comprising: a main part which has an external form of an epiphysis approximately fitting an internal form of the insertion hole, wherein stiffness around a boundary between epiphysis and diaphysis of the said main part varies so as to lower the stiffness as approaching the diaphysis; and a neck to place a spherical head in an artificial joint provided at a proximal end of the main part.”

Although a specific composition of the composite material for the stem in the invention does not need to be limited, the fiber-reinforced plastic can be used. As for the fiber, carbon fiber, ceramic fiber, glass fiber, aramid fiber can be exemplified. Turning the fiber into the continuous fiber, one can use it as filaments, blind shape, woven fabrics, and nonwoven fabrics, or turning into the short fiber, one can use it as chop shape. The carbon fibers are preferable and the high modulus carbon fiber is the most preferable among them. As for the resin, polyether ether ketone, polyetherimide, polyether ketone, polyacryl ether ketone, polyphenylene sulfide, polysulfone can be exemplified. The most preferable is the thermoplastic resin that is harmless to the human body and does not melt out.

In terms of the method of matching the stem's external shape with the internal shape of the insertion hole, although it is not limited to a specific composition, one can, for example, take pictures of several cross-sections of a patient's bone to which the stem is fixed, by using a nondestructive tomography scanner such as CT and MRI, and generate a numerical data after converting the said cross-sectional images to three-dimensional using three-dimensional CAD, and penetrated insertion hole with the prescribed internal shape into the patient's bone by the computer controlled surgical robot using the said numerical data. On the other hand, one can match the stem's external shape with the internal shape of the insertion hole by formulating the molding tool using the same numerical data and form the external shape of the stem based on the said molding tool.

Furthermore, as for the method of changing the stiffness of the stem's main part, although it is not limited to a specific composition, the stiffness can be changed, for example, by formulating the stem with the composite material with the prescribed thickness and making the thickness thinner as approaching from the epiphysis area to the diaphysis area. Or the stiffness can be changed by changing the fibrous direction of the reinforced fiber included in the composite material. Also, the stiffness can be changed by reducing the reinforced fiber's portion in the composite material, volume, or quantity as approaching from the epiphysis area to the diaphysis area. Moreover, the stiffness can be changed by reducing the elastic coefficient of the reinforced fiber in the composite material as approaching from the epiphysis area to the diaphysis area. These methods can be used separately or in combination, and it is not limited to these examples so long as the stiffness can be changed.

According to the invention, the gap between the stem and bone can be reduced as much as possible since the stem's external shape fits the internal shape of the insertion hole that is penetrated into bone. As a result, the stem can be well connected to bone with the use of cement, and there is no adverse affect on the human body through the melt-out of the unreacted monomer from not being mixed enough or the mixture ratio is inaccurate, as in a case of cement-type stem.

Also, because the stem's external shape fits the internal shape of the insertion hole that is penetrated into bone, the initial fixation adequate for a normal life style can be attained in the early postoperative period, and because the rotational anchorage is high, an early discharge from hospital is possible through shortening the hospitalization period and an early social rehabilitation is possible, which could relieve some of the burden from patients. Also, this method can be utilized to the elderly, who have concerns about adverse effects to the motor functions and other functions resulting from a long-term hospitalization.

Furthermore, because the stem's external shape fits the internal shape of the insertion hole that is penetrated into bone, fit and fill can be high, and the loading from the stem can be transferred to bone without deviation, and the stress shielding can be controlled, and the stem not getting loose, through weakening of the connection between the stem and bone as a result of stress shielding that makes bone skinnier, can be prevented and the stem's durability increases.

Moreover, because the stem's external shape fits the internal shape of the insertion hole that is penetrated into bone, the stem can be fixed without slamming the stem into the insertion hole with a hammer, and the stem can be utilized for osteoporosis patients and elderly people whose bone's strength is weak.

Also, because the stem's external shape in the epiphysis area fits the internal shape of the insertion hole that is penetrated into bone, fit and fill can be high, and the stem can be fixed in the epiphysis area. That is, using an example of the femur, as the epiphysis area, the stem can be fixed near the femur, which means the proximal fixing is possible, transferring the loading well from the stem to bone.

Also, in the proximity of the boundary between the epiphysis area and the diaphysis area, the stiffness of the stem's main part varies in such a way that the stiffness becomes low as approaching toward the diaphysis. As a result, the stress concentration at the ends of the connecting section between the stem's main part and bone can be controlled, and the stem getting loose because of the stress concentration that breaks away the connecting section can be prevented. Also, since the stiffness in the diaphysis area is made low, the stem's loading is mainly transferred to the epiphysis area. If applied to the femur, for example, the proximal fixing, in which the force is transferred in the epiphysis area that is the proximal side, can be done.

Furthermore, the composite material is used as the stem's material, in particular, by using the composite material that is harmless to the human body, there is no adverse affect to the human body unlike the conventional metallic stem in which the harmful substance to the human body melts out from the stem to the inside of human body. Also, the composite material is excellent in formability and workability compared to the titanium alloy, and the desirable shape can easily be attained, which reduces the cost of producing the stems.

The cement-less type artificial joint stem with the use of composite material further comprising “a guide section, provided at the tip of the main part and placed at the disphysis, the guide section has a lower bending and stretching/tensile stiffness than the main part.”

According to the invention, the guide section is provided in the forefront of the stem, and as a result, the stem can be easily inserted in the insertion hole during the operation when inserting the stem into the insertion hole penetrated into bone because the stem's insertion is guided by the guide section.

Also, since the bending and tensile stiffness of the guide section is made lower than the main part, the stress applied to the connecting section between the guide section and bone can be less than the main part. To elaborate, the stem in the invention has the same composition as the example shown in FIG. 21(D). That is, the left side of the figure, which includes the taper section 124 of the member 123, is equivalent to the stem's main part, and the right side of it is equivalent to the guide section, the member 120 is equivalent to bone, as well as the adhesive section connecting the member 123 and member 120 is equivalent to the connecting section between the stem and bone. As a result, the stress concentration at the ends of the connecting section between the stem's main part and bone can be controlled, and may prevent the stem from getting loose due to the stem's separation from bone. Also, the stem's loading is transferred from the guide section to bone via the main part, thus for the femur, for example, it is the proximal fixing and the stem's loading can be well transferred to bone. Furthermore, also at the guide section, the stress shielding can be controlled for bone contacting the guide section, since the compression stress is equally applied.

The cement-less type artificial joint stem with the use of composite material in the invention can also have a composition that “clearance of a predetermined quantity between an external surface of the guide section and an internal surface of the insertion hole is reserved.”

According to the invention, the loading is not transferred through the guide section since the clearance is formed between the insertion hole and the guide section and the guide section does not contact bone. That is, the fit and fill of the stem is low in the diaphysis area where the guide section is, and the stem is not fixed in this area but is fixed in the epiphysis area where the main part is, and thus the loading from the stem can be transferred to bone as a good condition.

Also, due to bone's growth after the surgery, even if the clearance between bone and the guide section is filled, it is filled with the cancellous bone that has low strength, making the stress applied at the connecting point with the guide section small. The loading from the stem is significantly applied in the epiphysis area where the main part is, and the anchorage in the epiphysis area is continuously maintained, and the loading from the stem can be transferred to bone in a good condition.

The cement-less type artificial joint stem with the use of either one of composite material, wherein “an external surface corresponding to the epiphysis has a convexo-concave surface treatment section thereon.” The surface finishing part can be a continuous convexo-concave shape, or can have the intaglio and convexity in several places on the flat surface, or can be provided with the adhesive line that includes the hydroxyapatite. These can be used separately or in combination, and the surface finishing part is not limited to these mentioned above.

According to the invention, the convexo-concave surface treatment section is provided in the external surface of the stem, and the mechanical bonding strength between the internal surface of the insertion hole and bone can be attained, and the anchorage strength adequate for a normal life style can be attained in the early postoperative period. As a result, it can relieve some of the burdens from patients who are hospitalized for a long time, and the stem in the invention can be utilized to elderly people.

Also, because the composite material is used for the stem in the invention, the surface finishing part can be provided more easily than the conventional stem, which used titanium alloy, a material that is difficult to be broken/cut. As a result, the stem's cost can be reduced even with the surface finishing part.

The cement-less type artificial joint stem with the use of composite material, wherein “the convexo-concave external surface treatment section has an adhesive layer containing hydroxyapatite on the most external surface, and fiber of composite material is positioned along with the convexo-concave external surface without breakage.” As for the hydroxyapatite, its crystal is preferable to use in order to increase the bonding strength.

According to the invention, since the hydroxyapatite crystal is included on the surface of the surface treatment section and the hydroxyapatite crystal and bone are chemically bonded, the stem and bone can be glued together more strongly in addition to the mechanical bonding by the convexo-concave of the surface finishing part.

Also, since the fiber form of the composite material are continuously provided inside along with the convexo-concave of the said surface treatment section, the fiber form of the composite material are continuous fibers and the strength of the composite material does not become low, and thus a high strength can be maintained.

Furthermore, since the composite material is used for the stem, the adhesiveness with the adhesive line, which includes hydroxyapatite, is better compared to the conventional stem of titanium alloy, and it is difficult for the stem to separate from the hydroxyapatite. Also, by using the resin for the adhesive line same as the resin used for the composite material, the adhesiveness becomes better between the adhesive line and the stem.

The cement-less type artificial joint stem with the use of either one of composite material, wherein “the main part comprises: a first external layer which contacts an internal surface of the insertion hole and has increased torsional stiffness; a main structure layer which is positioned inside the first external layer, continuing from the neck, and has increased bending stiffness; a core layer which is positioned inside the main structure layer and has lower stiffness than the main structure layer and the first external layer; and a most internal layer which is positioned between the core layer and the main structure layer.”

As for the method of increasing the torsional stiffness, the torsional stiffness can be increased by turning the direction of the composite material's fiber opposite of the torsional direction, for example, ±45° direction against the torsional direction. Also, as for the method of increasing the bending strength, the bending strength can be increased by turning the direction of the composite material's fiber perpendicular to the bending direction.

Also, in terms of the core layer with low stiffness, resin with non-reinforced fiber and plastic foam, or the composite material that uses short fiber can be used, and it is not limited to a specific material so long as its stiffness is lower than that of the main structure layer and the first external layer.

According to the invention, the main structure layer with strong bending stiffness is provided inside the stem and the first external layer with strong torsional stiffness is provided outside the stem. As a result, the stem's bending and torsional stiffness can be optimized.

The conventional stem was metallic such as titanium alloy, and its stiffness was unable to change in accordance with patients' condition, and thus the stem could not be used for the patients with weak bone as well as osteoporosis patients. However, according to the invention, the bending and torsional stiffness can be appropriately set up, and the stem can be adjusted to the characteristics of patients' bone in which the stem is to be filled. For example, for elderly people with weak bones and osteoporosis, the stem can be made in accordance with the stiffness of their bones. As a result, one can restrain a case in which bone is broken due to a significant difference in the stiffness of the stem and bone, and thus the stem can be applied to patients who had been unable to use the artificial joint.

As mentioned above, according to the invention, one can provide the cement-less type artificial joint stem with the use of composite material, which connects bones without using cement, not getting loose for a long period of time, excellent in the durability, and is provided with the stiffness and the external shape appropriate for each patient.

### BRIEF DESCRIPTION OF THE DRAWINGS

The above and other objects, features, advantages and technical and industrial significance of this invention will be better understood by the following detailed description of the preferred embodiments, when considered in connection with the accompanying drawings, in which:

FIG. 1 (A) is a front view of the artificial joint stem with the use of composite material of the invention, and (B) is the side view.

FIG. 2 (A) is the A1-A1 section view of FIG. 1, and (B) is the A2-A2 section view of FIG. 1.

FIG. 3 is section views of B1-B6 in FIG. 1 which are cut in each level perpendicular to the axes.

FIG. 4 (A) is an enlarged section view of the surface treatment section, and (B) is a further enlarged section view of the B part shown with an arrow in (A).

FIG. 5 (A) is a graph of the contact ratio to the cortical bone and the filling ratio in the medullary canal of the stem in FIG. 1, (B) is a graph of bending and tensile stiffness, and (C) is a graph of torsional stiffness.

FIG. 6 (A) is a front view of the other embodiment's stem of the invention, and (B) is the side view.

FIG. 7 is section views of C1-C6 in FIG. 6 that are cut in each level perpendicular to the axes.

FIG. 8 (A) is a graph of the filling ratio in the medullary canal of the stem in FIG. 6, and (B) is a graph of bending and tensile stiffness, and (C) is a graph of torsional stiffness.

FIG. 9 (A) is a front view of the other embodiment's stem of the invention, and (B) is the section view.

FIG. 10 shows the structure of the conventional total hip prosthesis.

FIG. 11 is top views showing the examples of the conventional metal-made cement-type total hip prosthesis stem.

FIG. 12 (A) shows the condition before the cement-type total hip prosthesis stem is placed, and (B) is the section view, showing the condition in which the stem is placed in the femur.

FIG. 13 is a section view of the internal structure of the epiphysis in the proximal side of the femur.

FIG. 14 is an enlarged section view of the internal structure of bone.

FIG. 15 (A) is a graph, showing the relations between the bone's modulus ratio and the average porosity of bone, and (B) is a graph, showing the relations between the thicknesswise compression ratio of bone and the average porosity of bone.

FIG. 16 is top views showing the examples of the conventional cement-less type total hip prosthesis.

FIG. 17 (A) shows an enlarged view of the principal part of convex portion on the side of stem, and (B) is a fragmentary sectional view of the further enlarged sectional view.

FIG. 18 is a section view of the conventional cement-less type total hip prosthesis stem fixed to the femur and cut in the axial direction, which is a different embodiment from that of FIG. 16.

FIG. 19 is section views of Z1-Z13 in FIG. 18 that are cut in each level perpendicular to the axes.

FIG. 20 is a graph, showing the change of fatigue strength by cyclic loading of composite material and titanium alloy.

FIG. 21(A) shows the condition of stress on the adhesive joint when members of the same stiffness are glued together, (B) shows the condition of stress on the adhesive joint when members of different stiffness are glued together, (C) shows the condition of stress on the adhesive joint when the length of the adhesive joint of the example (B) is shortened, and (D) shows the condition of stress when the stiffness of either member is changed on the way.

### DESCRIPTION OF THE PREFERRED EMBODIMENTS

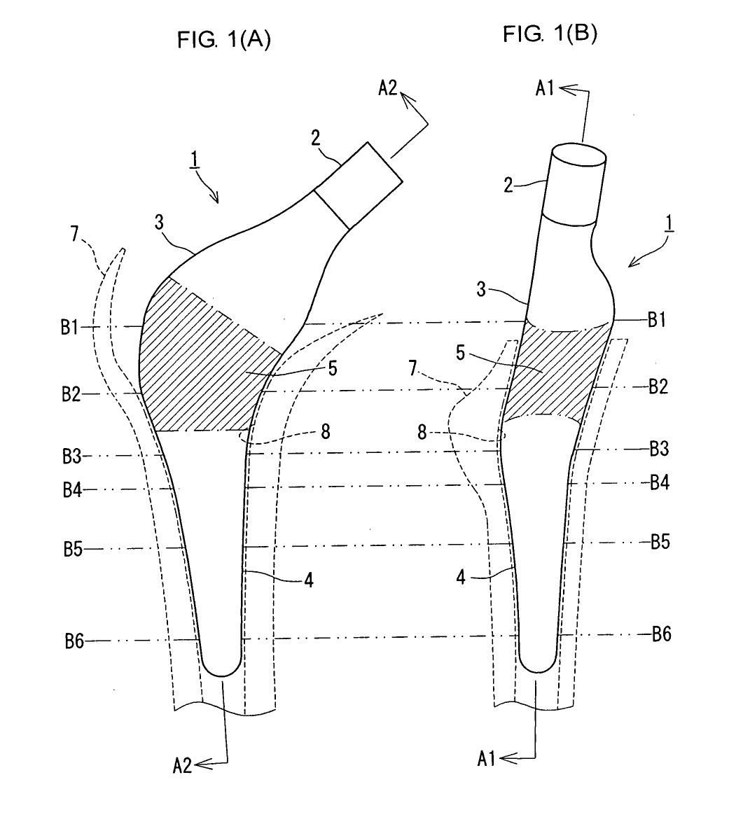

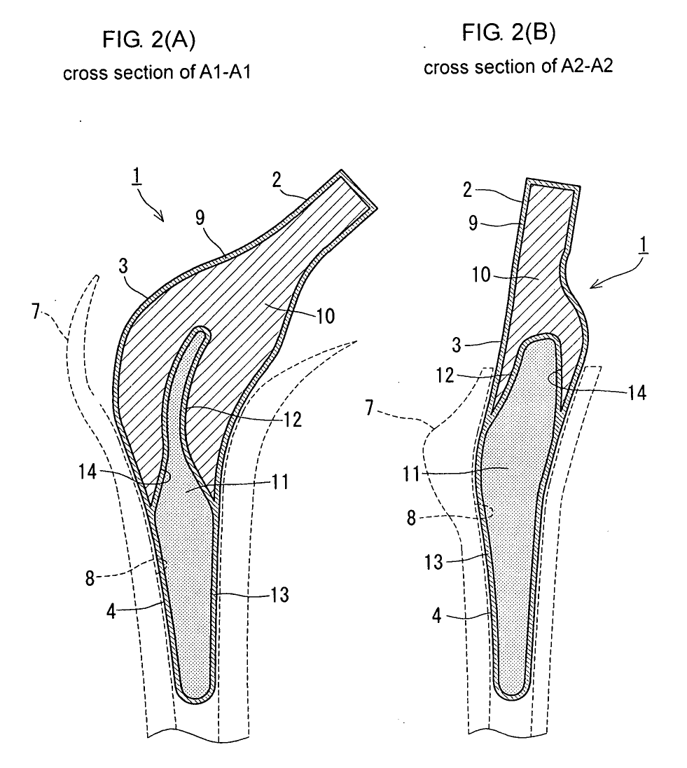

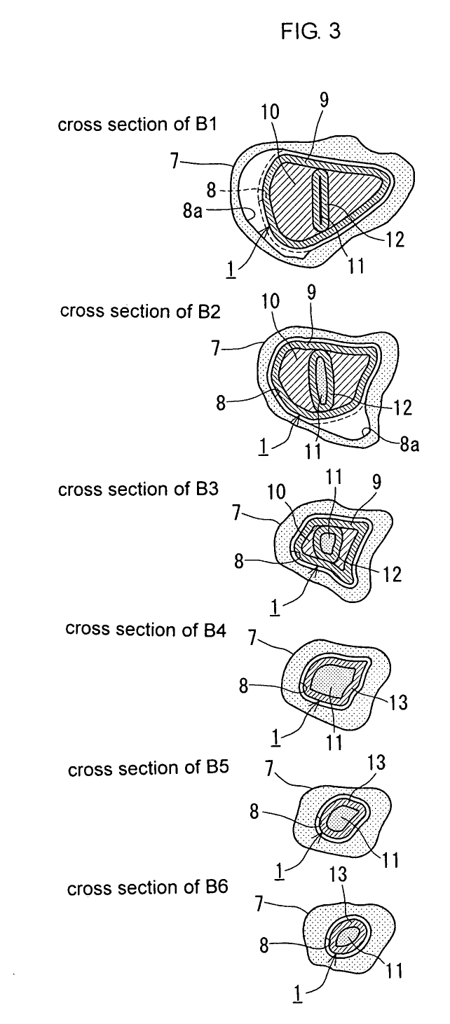

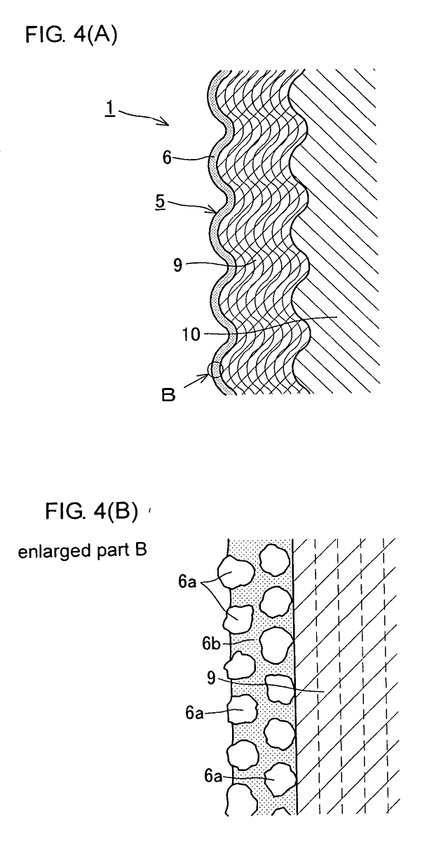

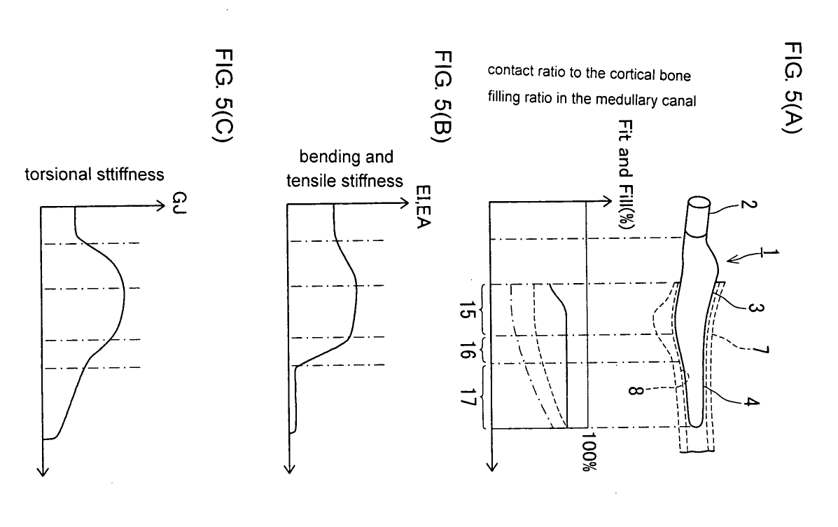

Below, the preferred embodiments are illustrated in details based on the FIGS. 1 through 5. FIG. 1(A) is a front view of the cement-less type artificial joint stem with the use of composite material in the invention, and FIG. 1(B) is its side view. FIG. 2(A) is the section view A1-A1 in FIG. 1, and FIG. 2(B) is the section view A2-A2 in FIG. 1. FIG. 3 is section views of B1-B6 in FIG. 1 that are cut in each level perpendicular to the axes. FIG. 4(A) is the section view showing the enlarged structure of the surface treatment section, FIG. 4(B) is a section view of the further enlarged B part shown with an arrow in FIG. 4(A). Also, FIG. 5(A) is a graph showing the contact ratio to the cortical bone and the filling ratio in the medullary canal, and FIG. 5 (B) is a graph showing the bending and the tensile stiffness, and FIG. 5 (C) is a graph showing the torsional stiffness.

As shown in FIG. 1, the artificial joint stem in the example is the artificial stem for the hip joint to be fixed in the femur. The stem 1 is made of the composite material and is comprised of the neck part 2, the main part 3, and the guide section 4. The neck part 2 is provided at a base end part of the stem 1, and an unshown spherical head is fixed thereon. The main part 3 is fixed on the femur, and the guide section 4 is adjacent thereto.

The surface finishing part 5 is formed at the main part 3 of the stem 1, provided with concave-convex on the part of its surface. Further, as shown in the enlarged view of FIG. 4, the chemically bonded layer 6 is formed by impregnating the hydroxyapatite crystal 6a in the plastic film 6b using as the adhesive agent and bonding thereto. By the convexo-concave of the surface finishing part 5, the mechanical bonding is made high between the stem 1 and the insertion hole 8 penetrated into bone 7 for the stem 1 to be embedded. Also, the chemical bonding with bone 7 is made high with the hydroxyapatite crystal 6a which is impregnated in the chemical bonding layer 6 of the surface, allowing the stem 1 to be glued together with the bone 7 more firmly. The chemical bonding layer 6 is equivalent to the adhesive layer in the invention.

As shown in FIG. 2, the internal structure of the stem 1 is configured to have the first external layer 9 with increased torsional stiffness which contacts with the internal surface of the insertion hole 8 penetrated into the bone 7, the main structure layer 10 with its increased bending stiffness which is placed inside the first external layer 9 and is subsequent from the neck part 2 to the main part 3, and the core layer 11 with lower stiffness than the main structure layer 10 and the first external layer 9 that is positioned inside the main structure layer 10, the inner most layer 12 that is placed in between the core layer 11 and the main structure layer 10, and the second external layer 13, which forms the external surface of the guide section 4, with lower stiffness than the structure layer 10 and the first external layer 9.

The composite material used for the stem 1 is the carbon fiber reinforced plastic. As for the carbon fiber, the high modulus, high strength carbon fiber with its elasticity of 200-650 GPa, for example, is used. Also, as for the matrix, the thermoplastic resin, such as polyether ether ketone and polyetherimide which are harmless to the human body, is used. The sizing can be applied to the carbon fiber in order to increase the bonding strength to the matrix. Incidentally, as for the stem 1 in the example, if the carbon fiber with its elasticity of 630 GPa used and the layer with its fiber direction ±45° is formed, the layer's transverse modulus G is about 49 GPa, which has enough strength when comparing to the conventional titanium stem of 43.3 GPa.

For the first external layer 9 of the stem 1, the fiber form of the composite material are woven fabric, and the direction of the fibers is directed ±45° to the axis of the main part 3 of the stem 1. As a result, the torsional stiffness increases and the shear loading and the torsional loading that are applied to the stem 1 can be supported at the first external layer 9.

Also, for the main structure layer 10 of the stem 1, the fiber form of the composite material are woven fabric, and the direction of the fibers is directed toward the axis of the main part 3 of the stem 1. As a result, the bending stiffness increases and the bending loading that is applied to the stem 1 can be supported at the main structure layer 10.

As shown in FIG. 2 (A), this main structure layer 9 is extended from the neck part 2 to the forefront section of the main part 3. That is, it is extended to the boundary between the epiphysis area and the diaphysis area of the bone 7, while the stem 1 is being fixed on the bone 7. Further, the core 11 goes inside of the main structure layer 10 through a given depth from the side of the guide section 4 of the stem 1.

Furthermore, the taper part 14 is formed in the internal edge of the main structure layer 10, as a result of the core layer 11 going into the main structure layer 10. The thickness of the main structure layer 10 is varied on the taper part 14, and the stiffness of the main structure layer 10 is changed at the taper part 14. In this case, the stiffness in the main structure layer 10 gets lower toward the forefront side.

The core layer 11 of the stem 1 is formed with the low-stiffness material such as plastic foam, and both the inner most layer 12 and the second external layer 13 are made with the low-stiffness material or the layers with its fibers directed at ±45°. The stiffness of the core layer 11 and the second external layer is the minimum required stiffness necessary to insert the stem 1 into the insertion hole 8 in the operation.

As for the stem 1, as shown in the section views B1-B6 in FIG. 3, the external shape of the stem 1 fits to the internal shape of the insertion hole 8 (the medullary canal 8a) penetrated into the bone 7 in most of the cross sections perpendicular to the axis.

The production method of the stem 1 in the example is explained next. First of all, several cross-sectional images of the patients' bone, on which the stem 1 is fixed, are captured, by using a nondestructive tomography scanner such as CT and MRI, and a numerical data after converting the said cross-sectional images to three-dimensional is generated by using three-dimensional CAD. Then the insertion with the prescribed internal shape (the internal shape of the medullary canal is preferable) is penetrated into the patient's bone by the computer controlled surgical robot using the said numerical data. On the other hand, the molding tool is manifactured using the same numerical data, and manifacture the stem 1 using the molding tool (not shown in a figure).

In formulating the stem 1, the plastic film that is impregnated with hydroxyapatite crystal is placed at the surface treatment section 5 of the molding tool, and the woven fabric, which is made with the carbon fiber that forms the primary surface layer 9 and the fiber made by thermoplastic resin, is laid up therein. At this time, the direction of the woven fabric's fibers is ±45° to the axis of the stem 1.

Furthermore, the woven fabrics, which is made with the carbon fiber and the fiber made by the thermoplastic resin those forms the main structure layer 10, are laid up in such a way that the fiber's direction faces toward the axis of the stem 1. The woven fabric that forms the inner most layer 12 and the second external layer 13 is laid up, and the plastic foam to be the core layer 11 is placed in the space formed by the inner most layer 12 and the second external layer 13.

Next, close the forming die, and give heat and pressure it using the autoclave or hot plate. When doing so, the pressurization can be done from the inside the stem 1 as a result of the plastic foam that forms the core layer 11 being expanded by the heat.

The convexo-concave that forms the surface treatment section 5 is engraved in the surface of the forming die of the stem 1, and the surface finishing of the part 5 is created while the stem is made. As shown in FIG. 4, the first external layer 9 and the main structure layer 10 inside of the surface finishing of the part 5 is also formed along with the shape of the surface finishing 5 without its fiber cut.

As shown in FIG. 5(A), while the stem 1 processed in the way mentioned above has the low contact ratio to the cortical bone and the filling ratio in the medullary canal, that is fit and fill, near the opening of the insertion hole 8, the fit and fill is higher in the more forefront side, and undergoes the transition at about 70% contact ratio to the cortical bone and filling ratio in the medullary canal all the way to the forefront side (side of the guide section 4).

FIG. 5(A) is the contact ratio to the cortical bone and filling ratio in the medullary canal shown in the form of a graph (solid line), and its contact ratio to the cortical bone and the filling ratio in the medullary canal are significantly higher than the conventional cement-less type stem (a dashed lines) and the custom made stem (dashed lines) in which the conventional cement-less type stem is improved. That is, the fit and fill of the stem 1 is generally high in the main part 3 and the guide section 4. The reference number 15 in the figure is the area where the main body 3, in which the taper part 14 is not provided, is located. The reference number 16 is the area where the taper part 14 of the main part 3 is provided. The reference number 17 is the area where the guide section 4 is located.

However, as shown in FIG. 5(B) and FIG. 5(C) of the same figure, in the epiphysis and the diaphysis area, that is the part in the main structure layer 10 of the stem 1 where the taper part 14 is provided, the bending and tensile stiffness are quickly decreasing and the torsional stiffness is gradually decreasing, as getting toward the forefront side (the side of the guide section 4) of the stem 1. As a result, because the stiffness of the guide section 4 is low although the overall fit and fill is high, and the stem's loading is transferred to the bone 7 through the high-stiffness main part 3, the proximal fixing of the stem 1 is possible.

This is also illustrated in FIG. 3. To elaborate, from this cross section, in the main part 3, the main structure layer 10 is mainly occupied, and the bending and tensile stiffness is granted by the main structure layer 10 and the first external layer 9 outside of it. And the low-stiffness core layer 11 and the internal layer 12 are expanded to the center of the stem 1 as getting from the main part 3 to the guide section 4, and there are only low-stiffness core layer 11 and the second external layer 13 at the guide section 4. From this, we know that the loading of the stem 1 is largely transferred to bone 7 at the main part 3.

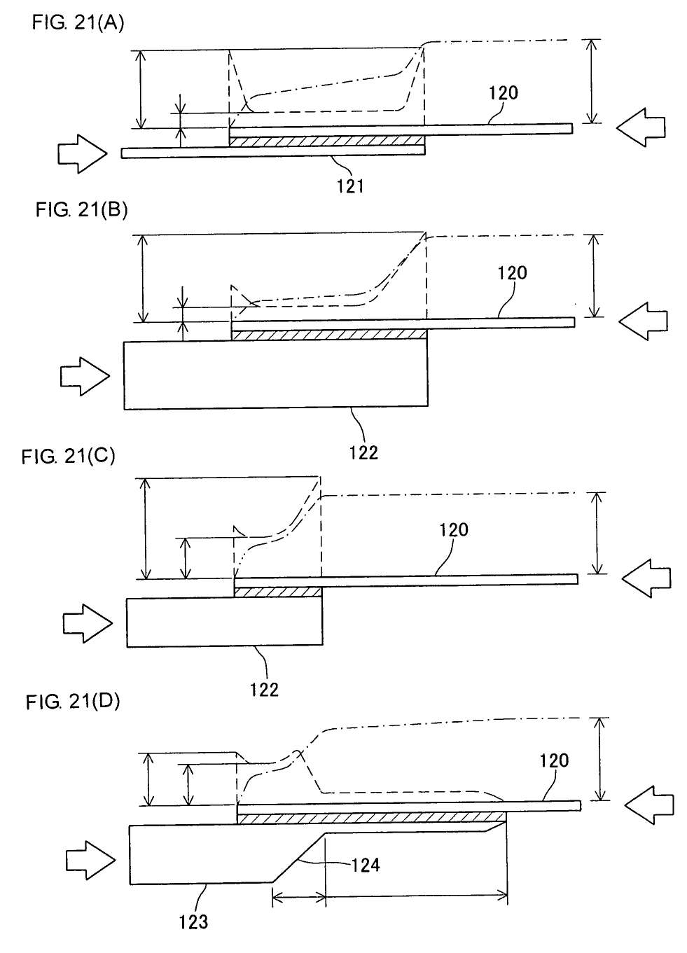

The load transfer concept between the stem 1 and the bone 7 is the same as the one shown in FIG. 21 (D), thereby the stress concentration on the both ends of the contact layer of the bone 7 is restrained.

As such, according to the figure in this operation, because the external shape of the stem 1 fits the internal shape of the insertion hole 8 penetrated into the bone 7, the gap between the stem 1 and the bone 7 can be reduced as much as possible. As a result, despite the cement-less type, the initial fixation adequate for a normal life style can be attained in the early postoperative period, and because the rotational fixation is high, an early discharge from hospital is possible through shortening the hospitalization period, and an early social rehabilitation is possible and thus relieving the burden on the patient. Also, this method can be utilized to senior people, who have concerns about adverse effect of motor functions and other functions resulting from a long-term hospitalization.

Also, because the external shape of the stem 1 fits the internal shape of the insertion hole 8 penetrated into bone 7, the fit and fill can be high and the loading from the stem 1 can be transferred to the bone without deviation, and therefore the stress shielding can be controlled and loosening the stem 1, due to weakening of the connection between the stem 1 and the bone 7 as a result of stress shielding that makes the bone 7 thinner, can be prevented, thereby increasing the artificial joint's durability.

Furthermore, by providing the taper part 14 in the main structure layer 10 of the main part 3 of the stem 1, the stiffness changes in such a way that the stiffness becomes low as getting toward the forefront side of the stem 1. As a result, the stress concentration can be controlled at the end of the contact layer between the bone 7 and the main part 3 of the stem 1, preventing the stem 1 from getting loose through separating the contact layer by the stress concentration. Also, since the stiffness in the diaphysis area is made low, the loading from the stem 1 is mainly transferred to the epiphysis area. That is, the proximal fixing can be done.

Also, since the guide section 4 is provided in the forefront side of the stem 1, and the insertion of the stem 1 is guided by the guide section 4 when the stem 1 is inserted into the insertion hole 8 penetrated into the bone 7 during the operation, the stem 1 can be easily inserted into the insertion hole 8.

Furthermore, since the surface treatment section 5 with convexo-concave is provided on the surface of the main part 3 and the chemically joint layer 6 having the hydroxyapatite crystal further above it, the mechanical and chemical bonding between the stem 1 and the bone 7 are possible, making it a stronger bonding, and thus the stem 1 can be prevented from getting loose.

Also, the composite material is used for the stem 1, which has better shape formability and the workability compared to the one made of the metals, and thus the production cost of the stem 1 can be reduced. Furthermore, the surface treatment section 5 and the stem 1 are formed simultaneously, and no additional process for providing the surface finishing part 5 is necessary, and thus enabling to control a rising cost even if the surface finishing part 5 is provided with the stem 1.

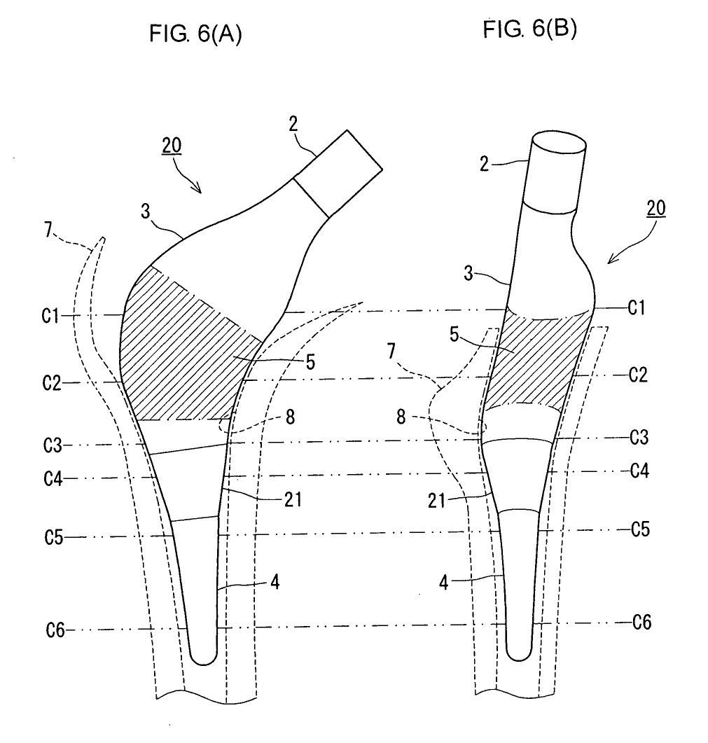

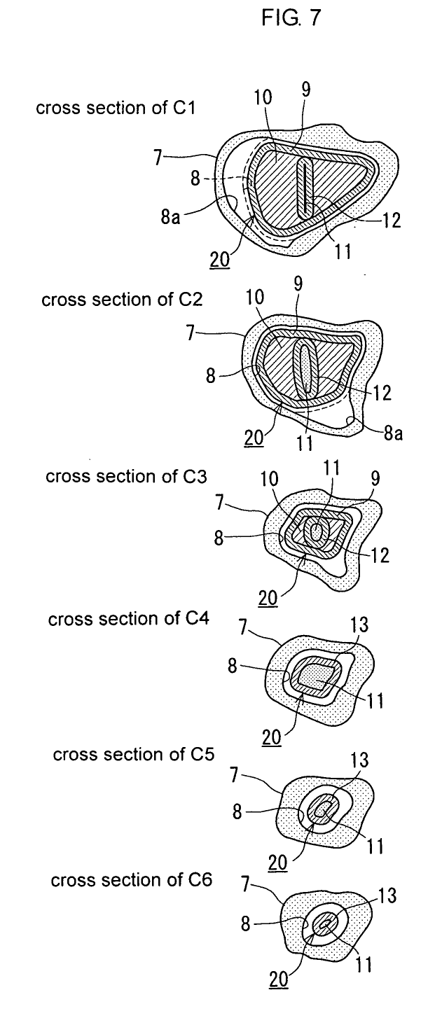

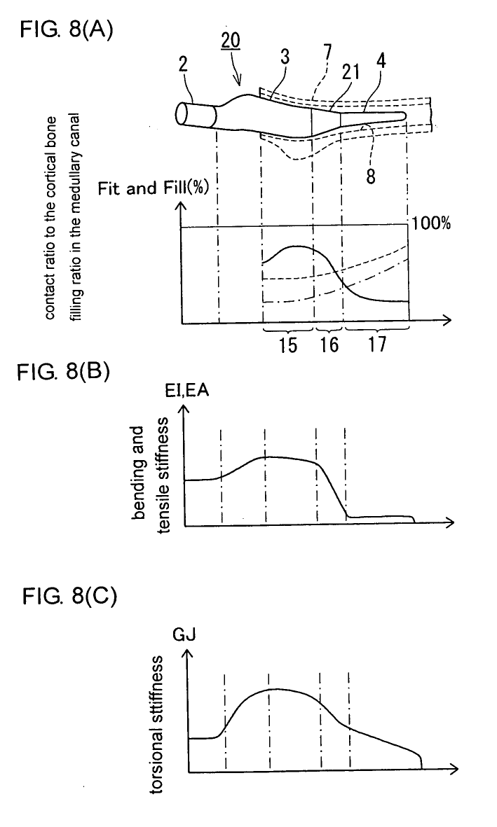

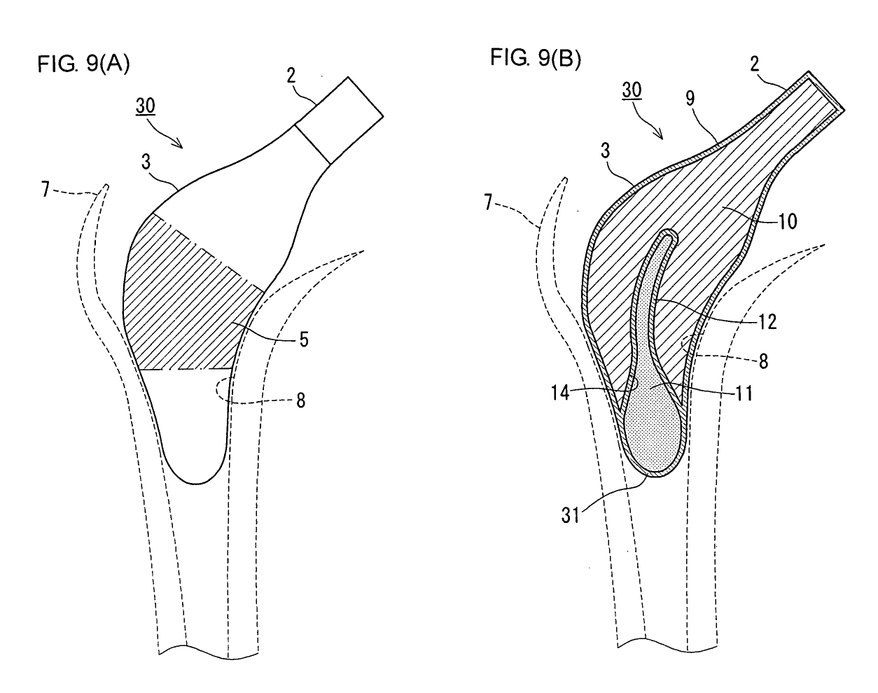

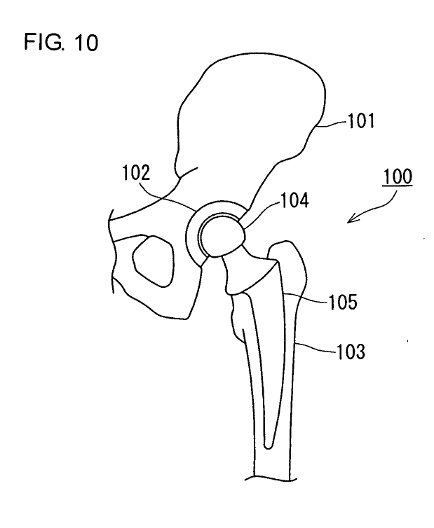

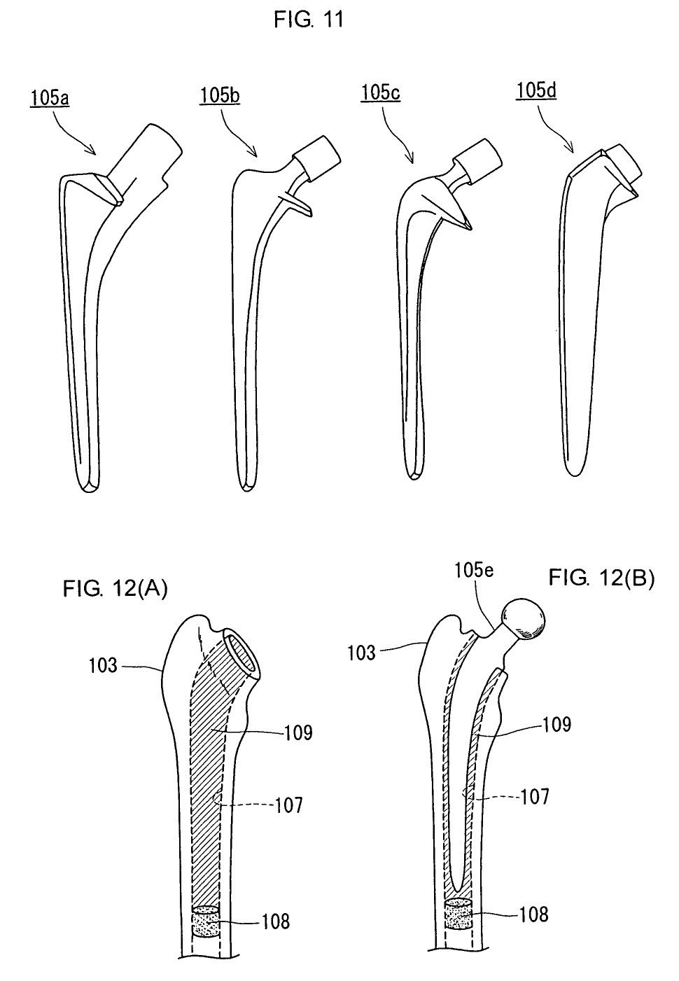

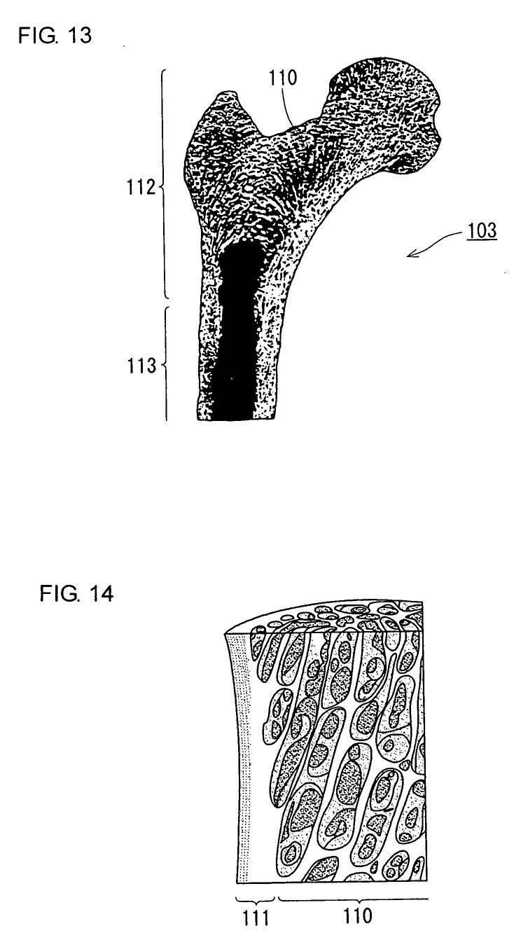

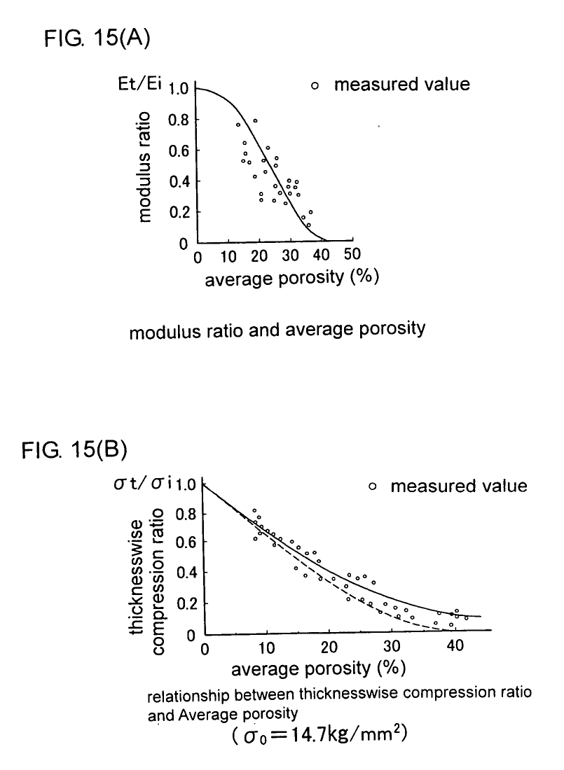

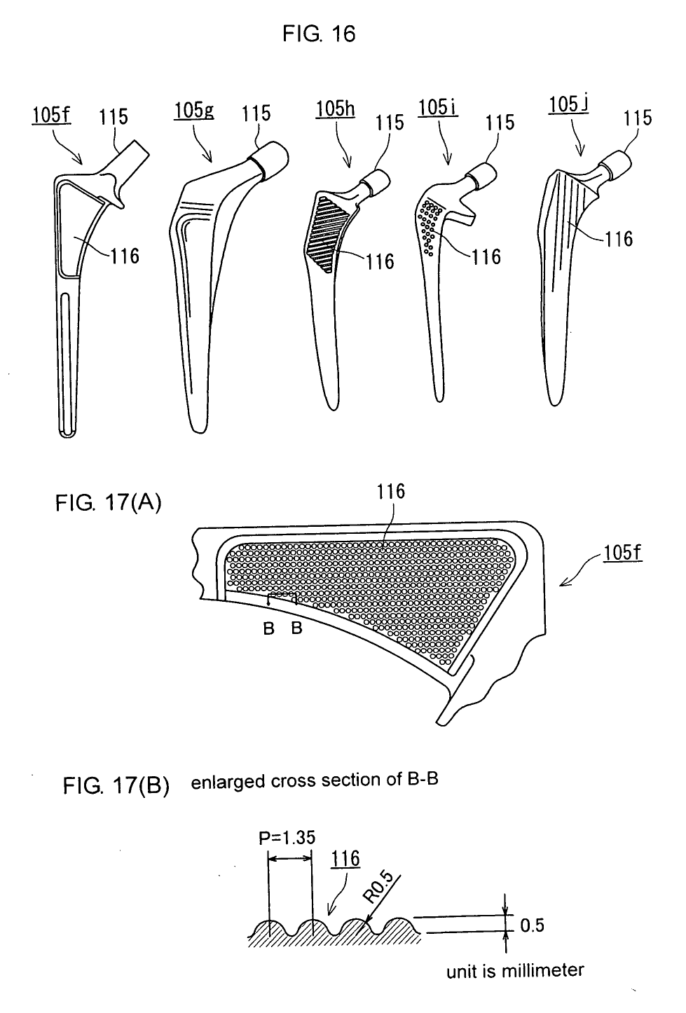

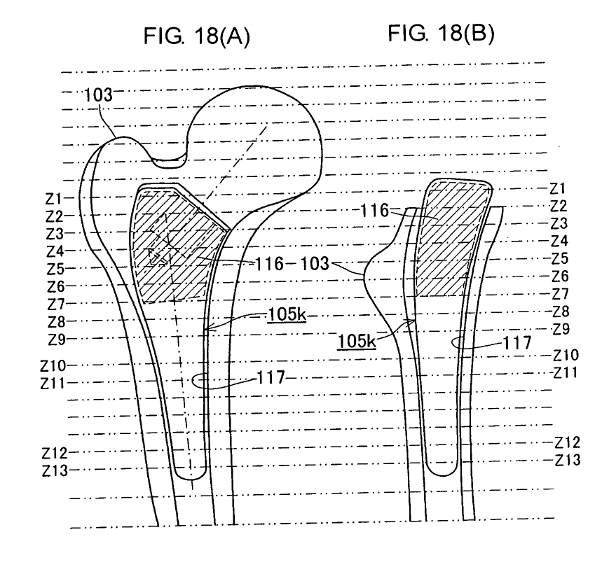

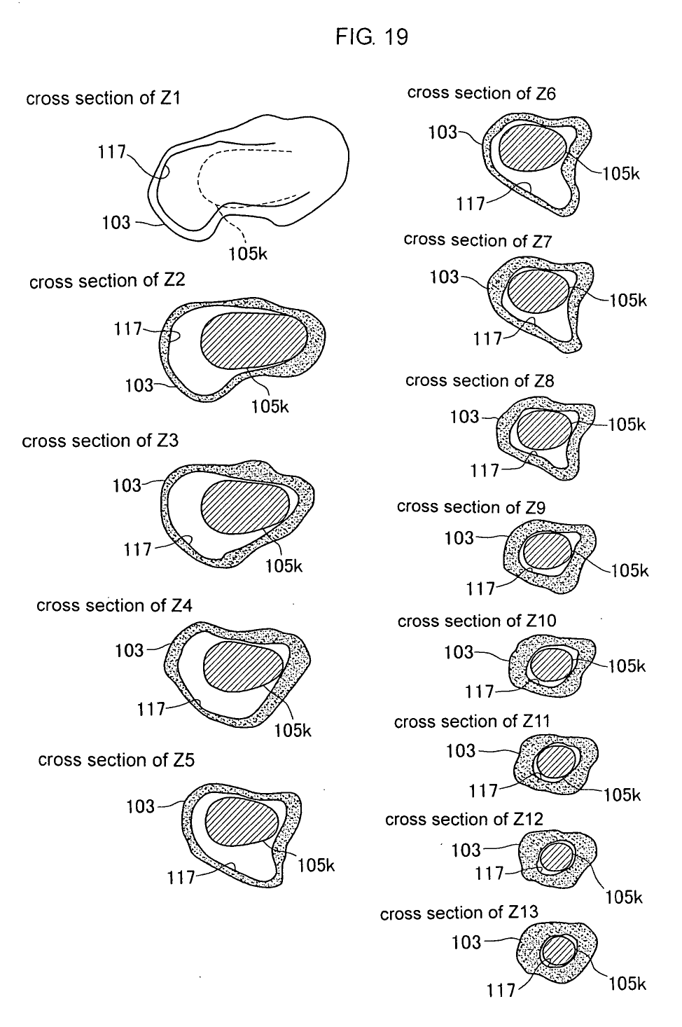

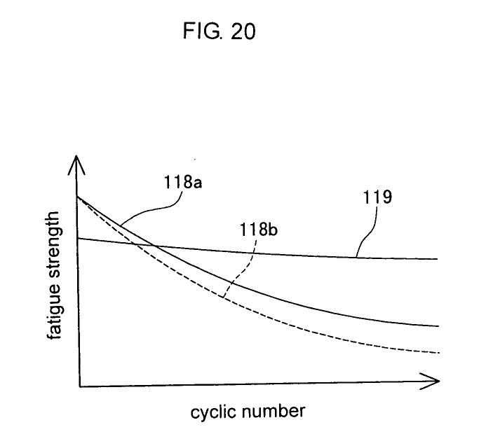

Next, we will describe the artificial joint stem with the use of composite material with an embodiment different from the ones mentioned above using FIGS. 6-8. FIG. 6(A) is the front view of the stem with another embodiment of the invention, and FIG. 6(B) is its side view. FIG. 7 is section views of C1-C6 in FIG. 6 that are cut in each level perpendicular to the axes. Also, FIG. 8(A) is the graph showing the contact ratio to the cortical bone and the filling ratio in the medullary canal, and FIG. 8(B) is the graph showing the bending and the tensile stiffness, and FIG. 9(C) is the graph showing the torsional stiffness. As for the parts similar to the abovementioned example, the same signs are provided and the detailed illustration is omitted.

The stem 20 in this embodiment has a high fit and fill at the main part 3, that is, in the epiphysis area, and a low fit and fill at the guide section 4, that is, in the diaphysis area, making a perfect anchorage between the stem 20 and the bone 7 in the epiphysis area, that is, the proximal fixing.

As shown in FIG. 6 and FIG. 7, the taper part 21 is provided between the main part 3 and the guide section 4 for the stem 20 in this example, and the given amount of clearance is formed between the outer surface of the guide section 4 and the internal surface of the insertion hole 8, as a result of the external shape of the guide section 4 being smaller by the taper part 21.

From this, as shown in FIG. 8 (A), while the contact ratio to the cortical bone and the filling ratio in the medullary canal (fit and fill) are high in the main part 3 of the stem 20, the fit and fill decreases in the taper part 21, and the fit and fill for the guide section 4 remains low through the forefront.

As such, according to this embodiment, since the appropriate amount of clearance is formed between the external surface of the guide section of the stem 20 and the internal surface of the insertion hole 8, the guide section 4 does not contact with bone 7 in the early postoperative period, thereby the loading is not transferred to bone 7 through the guide section 4.

Also, after the surgery, even if the clearance with the guide section 4 is filled due to the growth of the bone 7, this part is filled with the low density cancellous bone, and the stress applied to the joint section with the guide section 4 is small, and the loading from the stem 20 is largely applied in the epiphysis area where the main part 3 is located. The anchorage in the epiphysis area is continuously maintained, and thus the loading from the stem 20 can be transferred to the bone 7 in a good condition.

Furthermore, as for the stem 20 in this example, since the guide section 4 is thin, the friction of the guiding 4 is low when the stem 20 is inserted into the insertion hole 8 during the surgery, and thus the insertion can be done more easily than the stem 1 in FIG. 1.

Another embodiment of the invention using FIG. 9 will be illustrated next. FIG. 9(A) is the front view of the stem of the further embodiment, and FIG. 9(B) is its section view. The stem 30 of this embodiment has the characteristic of not having the guide section, and it is the embodiment of the stem 1 in FIG. 1 with the guide section 4 being deleted. The reference number 31 in the figure is the tertiary outer layer, covering the bottom of the core layer 11 at the bottom of the stem 30.

For the stem 30, similar to the system mentioned above, the stem 30 can be well fixed in the epiphysis area and provide the same effects as the one mentioned above. In this example, the core layer 11 and the tertiary outer layer 31 can be deleted and the main part 3 can be hollow shape.

So far, we have illustrated the various embodiments of the invention, yet the invention is not limited to these embodiments, and various improvements as well as changes of design are possible to the extent it does not deviate from the scope of the invention, as indicated below.

That is, in this embodiment, the carbon fiber reinforced thermoplastic such as PEEK and PEI are shown as the composite materials, yet it is not limited to these materials. For example, as for the fiber, the ceramic fiber, glass fiber, and aramid fiber can be used, and as for the ceramic fiber, the ceramic fiber having the titanium component with the silicon carbide as a main body, such as the product name “tirano fiber” can be exemplified. Also, as for the plastic, one may use polyether ether ketone, polyacryl ether ketone, polyphenylene sulfide, polysulfone, and these raw materials can be used appropriately in combination.

Also, in this embodiment, the carbon fiber of composite material used for the stem 1 and the stem 20 that are same as the fibers for the main part 3 and the guide section 4 are shown, yet it is not limited to these materials. One may use the high modulus fiber for the main part 3 and the low modulus fiber for the guide section 4, or may use the carbon fiber for the main part 3 and the low modulus glass fiber for the guide section 4, thus these materials are not restricted so long as the stiffness of the guide section 4 is lower than that of the main part 3.

Furthermore, in this embodiment, the inner most layer 12 is provided with the stem 1, 20 and 30, yet it is not limited to such, and the stem can be without the inner most layer 12. As a result, one may reduce the cost of the stem since the manufacturing process of the stem is reduced.

Also, in this embodiment, in FIG. 6, the taper part 21 is provided between the main part 3 and the guide section for the stem 20, and the appropriate amount of clearance is formed between the external surface of the guide section 4 and the insertion hole 8 of the bone 7, yet it is not limited to this. For example, the clearance between the insertion hole 8 and the guide section 4 can be the same as the clearance between the main part 3 and the insertion hole 8. That is, the internal shape of the insertion hole 8 can be shaped along with the external shape of the stem 20. From this, too, the same effects as the one mentioned above is resulted.

The invention can provide cement-less type artificial joint stems with the use of composite material which can be connected to bone without using cement, does not become loose over a long period of time, has excellent durability, and has appropriate external form and stiffness to each patient.

Also, the invention can be used not only for the total hip prosthesis of the femur illustrated in the embodiment, but for the implant to connect joints such as knee joint, shoulder joint and fractured bone or for the substitute of damaged bone by accidents or diseases.

1. A cement-less artificial joint stem with the use of composite material to be inserted in an insertion hole which is penetrated into bone and fixed to bone without using cement, comprising:
a main part which has an external form of an epiphysis approximately fitting an internal form of the insertion hole, wherein stiffness around a boundary between epiphysis and diaphysis of said main part which is formed that the ratio of members giving stiffness on the cross section area perpendicular to the axis decreases as approaching toward the diaphysis so as to lower the stiffness as approaching the diaphysis; and
a neck to place a spherical head in an artificial joint provided at a proximal end of the main part,
and a guide section which is positioned at the tip of the main part in the diaphysis and has lower bending and stretching stiffness than that of the main part, wherein
the main part has the first external layer with heightened twisting stiffness which contacts the internal surface of the insertion hole; the main structure layer with heightened twisting stiffness which is positioned in the first external layer and continues from the neck; the core layer with lower twisting stiffness than the main structure layer and the first external layer which is positioned inside the main structure layer; and the inner most layer which is positioned between the core layer and the main structure layer.

2. The cement-less artificial joint stem with the use of composite material of claim 1, further comprising
the main structure of the main part further comprises a taper part which thickness decreases as moving toward the direction of the diaphysis in the neighborhood of the boundary between the epiphysis and diaphysis.

3. The cement-less type artificial joint stem with the use of composite material of claim 1, wherein
clearance of a predetermined quantity between an external surface of the guide section and an internal surface of the insertion hole is reserved.

4. The cement-less type artificial joint stem with the use of either one of composite material mentioned in claims 1-3, wherein
an external surface corresponding to the epiphysis has a convexo-concave surface treatment section thereon.

5. The cement-less type artificial joint stem with the use of composite material of claim 4, wherein
said convexo-concave external surface treatment section has an adhesive layer containing hydroxyapatite on the most external surface, and
fiber of composite material is positioned along with the convexo-concave external surface without breakage.

6. (canceled)

7. The cement-less type artificial joint stem with the use of composite material of claim 2, wherein
clearance of a predetermined quantity between an external surface of the guide section and an internal surface of the insertion hole is reserved.
